(19) United States
(12) Patent Application Publication
(10) Pub. No.: US 2011/0027866 A1
(43) Pub. Date: Feb. 3, 2011

(54) CARRIER PIECE AND METHOD FOR PREPARING CULTURE MEDIA

(75) Inventors: Jonathan N Roth, Goshen, IN (US); Geoffrey N. Roth, Goshen, IN (US)

Correspondence Address: BOTKIN & HALL, LLP, 105 E. JEFFERSON BLVD., SUITE 400, SOUTH BEND, IN, 46601, US

(73) Assignee: MICROLOGY LABORATORIES, LLC, Goshen, IN (US)

(21) Appl. No.: 12/449,958

(22) PCT Filed: Mar. 25, 2008
(86) PCT No.: PCT/US08/03863

§ 371 Date: Sep. 4, 2009

Related U.S. Application Data
(60) Provisional application No. 60/896,967, filed on Mar. 26, 2007.

(51) Int. Cl. C12N 1/20; C12M 1/00

(52) U.S. Cl. 435/253.6; 435/283.1

(57) ABSTRACT

The method provides a simple, accurate and inexpensive way to prepare culture media for conducting microbiological analysis with an absolute minimum of facilities, extraneous equipment and time consuming procedures. The invention introduces the use of a carrier piece that is suitable for carrying a reactant material for combining with growth media. The carrier piece may be formed using (non-)porous, (non-)absorbent material, including paper, plastic, gum, fabric, or acetate. The carrier piece carries a single one or a combination of various reactant materials that are released into the medium or medium/test sample mixture upon contact therewith. The reactant materials may include, among other things, nutrients, inhibitors, including antibiotics and bile salts, enzyme substrates, and/or a catalyst for a gelling agent contained in the growth medium. The growth medium, the carrier piece with the appropriate reactant material(s), and the test sample can be easily packaged and sold separately or as a kit and used anywhere.

### BACKGROUND OF THE INVENTION

1. Field of the Invention

This invention relates to culture media and methods for producing the same and, more particularly, to a carrier piece and a method for preparing culture media using a carrier piece.

2. Description of the Background of the Invention

Generally, media used for growth of living cells, tissues, or organisms contain certain ingredients. These ingredients include water, nutrients (typically a carbon source, a nitrogen source, and smaller amounts of other essential elements), buffers, and often a gelling or solidifying agent. A considerable variety of different methods for producing such media are also known. Agar pour plates, for example, continue to be used in some laboratories in food and water analysis and basic research. This approach is better than the pre-poured, pre-gelled agar plate because the test sample is mixed with the medium. Thus, cellular growth may occur within the medium and not just on the surface where the test cells were inoculated. Specifically, the test sample is carefully measured and placed in the plate and heated nutrient agar is poured over the sample, swirled to mix the two, and then allowed to gel.

Analysis of the sample for determining the type and the amount of the organism present is generally done the same way regardless of how the culture media is prepared. Following appropriate incubation, which allows growth of the microbes in the test sample as individual colony forming units, the colonies may be identified, counted, and tabulated. Analysis is usually performed visually and may be done under lights having different wavelengths that highlight characteristic cell colonies. The sample preferably contains no reflective particles or materials because such matter may reflect the light and interfere with viewing.

One problem in the heat-and-pour agar method is the heat. The high temperature may kill many types of microbes. One alternate approach to the agar pour plate was invented by the inventor of the present invention and is disclosed in U.S. Pat. No. 4,241,186. Briefly, the inventor's previously patented method uses an absorbent support material impregnated with a metal salt solution to produce gelling of the liquid growth medium. The liquid growth medium contains a low methoxyl pectin, which reacts with the metal cation material in the pad so that when the liquid growth medium is poured over the support pad, gelling of the medium occurs in the container. The procedure may be performed at room temperature, which saves microorganisms from temperature shock. Technicians may obtain a more accurate assessment of the number and type of organisms as a result.

The main drawbacks to this method, however, reside in the support material or pad. The pad must be positioned in the container before adding the growth medium. The size, shape, and surface area of the pad, therefore, must be especially designed to correspond with the particular container size and cubic area. Otherwise, the amount of reactive substance in the pad may be amiss and the liquid medium won't completely gel, or may not gel smooth and evenly, which tends to frustrate an accurate count of cell colonies. These shortcomings are made more troublesome and inefficient by the fact that one side or one-half the surface area of the pad is in contact with the bottom of the container. Also, depending upon the color of the pad and/or material from which it is made, visual acuity of the sample may not be ideal. The cell colonies are often camouflaged by the pad.

Methods for impregnating the support pad with the cation material also tend to be underdeveloped, and the material is typically disproportionately dispersed within the pad. As a result, when the pad makes contact with the growth medium, air pockets develop inside the medium as it solidifies. Air pockets are commonly mistaken for microbial colonies, affect the light, obscure viewing results, and thus negatively impact the accuracy of test results. When the pad is formed from a net or mesh material, the resulting compartmentalization of the sample can help improve the conditions under which the sample may be studied; but, mesh material presents more complicated impregnation methods due to the decrease of surface area, and the cation material remains disproportionately distributed in/on the pad.

Another patented approach developed by this inventor involves applying a growth-compatible gel having a multivalent metal cation material to the bottom of a Petri dish. The liquid growth medium is then combined with the gel to produce gelling. This method is the subject matter of U.S. Pat. No. 4,282,317. While the '317 method offers the benefits of having a preformed component of the culture media set and ready for use in a Petri dish, the inventor has experienced several drawbacks with the method that the carrier piece and method of the present invention solve.

First, in order to evenly apply the gel layer to the bottom of the container and produce the ready-for-use gel containers without air bubbles requires sophisticated equipment. Design and tooling costs associated with such equipment limit the type and size of containers that can be used and add to the cost per unit. Second, the mass production of gelled containers must be conducted under sterile conditions that are expensive to maintain and lengthen production time. Third, after application of the gel layer, the sterile containers must be repackaged and sealed for storage and/or shipment. Post-packaging temperature changes affect the water in the gel causing condensation, freezing, and other ruinous consequences as a result.

Thus, there remains a need for an improved method for preparing culture media. Additionally, analysis of the resulting cellular bodies grown in the medium is quicker and less expensive if certain materials heretofore contained in the growth medium, such as basic nutrients, antibiotics or other inhibitors for cell type selection, for example, can be simply delivered into the medium in a more deliberate, controlled manner. Such a material delivery method where the carrier device has no visually residual effect on the growth medium and test sample mixture is also desirable.

### SUMMARY OF THE INVENTION

The present invention introduces the use of a carrier piece that is suitable for carrying a reactant material. The carrier piece may be (non-)absorbent or (non-)porous material and may carry a single one or a combination of various materials that may be released into or react with the growth medium or growth medium/test sample mixture when the carrier piece is brought into contact with the same. The reactant material(s) carried by the piece may be absorbed by the piece or applied to the surface of the piece as a coating. The reactant materials may include, for example, nutrients, inhibitors, including antibodies, enzyme substrates, catalysts, (i.e., a “hardener” for a gelling agent contained in the liquid growth medium), or ions, and/or compounds, among other things. The material(s) may be incorporated singly or in any combination deemed desirable for a particular test or to achieve specific final outcomes.

According to the invention, a method for preparing culture media includes: (i) providing a medium for cultivating microorganisms; (ii) providing a carrier piece suitable for carrying a reactant material. The reactant material is capable of causing a change to the medium when the carrier piece contacts the medium; and (iii) contacting the carrier piece with the medium. In one form of the invention, the medium may be an aqueous solution.

In another aspect of the invention, the method may include the step of mixing a test sample with the medium to produce a mixture and then contacting the carrier piece with the mixture. The reactant material may include nutrients for nourishing microorganisms that may be contained in the sample.

The medium may include a gelling agent, and the reactant material may be a substance capable of changing liquid growth medium from a liquid phase to a substantially solid or gel phase when the carrier piece contacts the medium. The method may include the step of allowing the carrier piece to remain in the medium for a predetermined time so that the medium accretes on a surface of the carrier piece and removing the carrier piece with an attached accretion from the medium.

In one form of the invention, the carrier piece may be formed from a transparent material.

In another aspect, the carrier piece is formed from a piece of soluble gum or resin. The carrier piece may dissolve when brought into contact with the medium.

In another form of the invention, a kit is provided for preparing a test culture medium. The kit includes a medium for cultivating microorganisms, and at least one carrier piece for carrying a reactant material. The reactant material is capable of causing a change to the medium or to microorganisms when the carrier piece contacts the medium.

One object of the invention is to provide new method for preparing culture media for cultivating microorganisms that is inexpensive and easy to do. Related objects and advantages of the invention will be apparent from the following description.

### DETAILED DESCRIPTION OF INVENTION

It should be understood that the carrier piece and method of the invention are not limited by the types and variations of the growth media that are formulated. Essentially any type and formulation of generally known and published growth medium may be used with this invention. The carrier piece and method of the invention are thus not limited by constituents such as the nutrients, buffers, inhibitors, and so forth that may be combined with or without the carrier piece to form the particular growth medium. To the extent, however, that it is desirable to produce a gelled culture medium according to the invention, the growth medium will include a gelling agent, and the carrier piece will include a corresponding “hardener” for reacting with the particular gelling agent(s) present in the growth medium.

“Hardener,” or “hardener compound” in this description means (i) a substance added to the solution containing the reactant material, in which case when it's applied on or in the carrier piece, it may help solidify the solution upon dehydration, (ii) a substance that serves as the reactant material itself, and/or (iii) in some test applications, a substance that causes a liquid growth medium to change to a solid or gel phase when a gelling agent is present in the growth medium.

The growth medium is formed by combining the desired substrates with a nutrient base medium. The nutrient base medium can be any culture medium known in the art for providing the maintenance and production of living cells. Generally, such media include nutrients, buffers, water, and sometimes a gelling agent. Possible gelling agents include agars, pectins, carrageenans, alginates, locust bean, and xanthines, among others.

### Examples of Growth Media

Examples of growth media that may be used with the invention for the growth of microorganisms include the following: Modified M-TEC medium for the growth and differentiation of E. coli and General Coliform Bacterial

Ingredients:

| | | |
|---|---|---|
| Proteose Peptone #3 | 5 | gm |
| Yeast Extract | 3 | gm |
| Dipotassium Phosphate | 0.3 | gm |
| Bile Salts #3 | 1 | gm |
| Chomogenic Enzyme Substrate Mix | 0.3 | gm |
| Gelling Agent | 17 | gm |

With respect to the M-TEC medium above, the ingredients are blended in one liter of hot, de-ionized water, and then the pH is adjusted with an aqueous solution of Potassium Carbonate so that a final pH of between 7.0 and 7.4 is achieved. This Medium is intended for the eventual isolation, differentiation and identification of E. coli and General Coliform bacteria. E. coli will grow in the medium and form colonies (typically 1-2 mm diameter), which appear a dark blue/purple color in ambient light. Other General Coliforms will grow and form colonies (usually 1-2 mm diameter), which appear a pink/red color in ambient light. Closely related non-coliform bacteria may grow as colorless colonies, while many other bacterial types do not grow due to the presence of the Bile Salts inhibitor, which may, in this example, be a reactant material carried by the carrier piece.

### A General Medium for Heterotrophic Plate Counts (General Bacterial Populations)

Ingredients:

| | | |
|---|---|---|
| Peptone Mix | 5 | gm |
| Beef Extract | 3 | gm |
| Dextrose | 2.5 | gm |
| Gelling Agent | 17 | gm |

According to example #2 above, the ingredients are blended into 1 liter of hot de-ionized water and the pH is adjusted with an aqueous solution of Potassium Carbonate so that a final pH of between 7.0 and 7.3 is achieved. This medium exists in liquid form at room temperature and is intended to recover virtually any type of heterotrophic bacteria that may be present in the test sample. The carrier piece used with this medium may, for example, include a reactant material that reacts with the gelling agent to change the medium from a liquid to a substantially solid or gel phase. Microbe colonies that form in the gel may vary in size (usually 1-3 mm diameter) and color (usually white or colorless). The colony-forming units are then identified and, if applicable, counted.

The invention is intended to provide a simple, accurate and inexpensive way to produce test culture media for carrying out microbiological analysis with an absolute minimum of facilities, extraneous equipment and time consuming procedures. Everything needed for testing can be easily handled and shipped separately or as a kit. Material costs are low, and shipping and handling costs are minimal.

In one commercial form of the invention, a culture medium and carrier piece test kit and procedure may include (i) a clear-walled container containing sterile liquid nutrient growth medium, (ii) at least one sterile carrier piece, preferably contained in a container to prevent damage and contamination, and optionally, (iii) a container holding the test sample, which may or may not include target microorganism(s). The test is completed by adding a measured amount of the test sample into the container of sterile liquid growth medium. The container is recapped and gently mixed, preferably swirled or inverted to form a liquid growth medium/test sample mixture. Next, the cap is removed, and a carrier piece is added to the liquid growth medium/test sample mixture. The cap is replaced. The container is placed in an upright position in an incubator or warm environment conducive to the growth of the particular target organisms that may be present in the test sample. Depending upon the application, solidification of the medium may occur, and colonies will grow as individual entities upon sufficient incubation time.

### Preparation of a Carrier Piece

Generally, the preferred material of the carrier piece is such that when it is placed in contact with the nutrient growth medium or growth medium/test sample mixture it will be transparent. That is, the carrier piece will have essentially the same refractive index as the material of the growth medium or growth medium/test sample mixture. The carrier piece may be (non-)porous and/or (non-)absorbent, but should be suitable for carrying the reactant material(s) introduced in/on it. The material from which the carrier piece is formed should also have sufficient integrity to maintain shape without unacceptable bending, folding or loss of basic shape when handled and introduced to the liquid growth medium or liquid growth medium/test sample mixture. Acceptable materials include paper, plastic, gum, glass, or fibrous materials. Favorable results have been obtained using clear acetate or polyester sheet material to form the carrier piece. By using a soluble gum, resin (e.g., guar gum, locust tree gum, Alginate gum, Agar, gelatin, silica) or like material, the carrier piece will essentially dissolve and not interfere with subsequent visual analysis of the test sample.

The material to be carried on/in the carrier piece may be applied by various techniques, including spraying or dipping the carrier piece. The material may be applied in a pure form or it may be mixed with another material to cause adequate adhesion or sticking onto the carrier piece. In any case, the active ingredient or reactant material must be soluble enough to readily migrate from the carrier piece into the growth medium or growth medium/test sample mixture when the carrier piece is in contact with the same. The material carried by the carrier piece is preferably dehydrated so that the finished carrier piece with reactant material can be stored and handled neatly without wetness, stickiness, or other less preferable properties. The reactant material is thus evenly distributed on/in the carrier piece, and the carrier piece eliminates air bubbles and other obfuscations in the sample.

In one preferred embodiment, clear sheets of suitable material, preferably acetate or polyester having a thickness of between about 0.1-1.0 mm are used to form the carrier piece. The carrier piece should be reasonably stiff or rigid so that its shape will endure when cured and introduced into the growth medium or medium/test sample mixture.

Next, a solution containing the reactant material is made and may include a substance(s) to enhance and promote coating ability on the carrier piece. One preferred basic reactant material solution includes Calcium Chloride, which serves as a hardener compound, and agar-agar. Substitutes for the agar-agar may include one of various gums, adhesives, gelatin, or other materials. The agar-agar, however, through experimentation, has produced desirable characteristics with respect to viscosity and adhesiveness. When these constituents are mixed with water and heated, the resulting substance may be spread, sprayed, or dipped onto the carrier piece. This formula may be applied to both surfaces of the carrier material yielding an even, uniform coating layer covering both surfaces that will solidify upon cooling and which can be dehydrated, resulting in a dry, flexible coating over the entire sheet. With respect to the soluble gum or resin material, it, too, may be dried to produce a carrier piece having the aforementioned properties. The finished product can then be die-cut to any size and packaged as desired.

The carrier piece may be used with numerous different sized culture-growth containers, which means that the culture-growth containers may be purchased by users in the field or on location, thus eliminating the cost of packaging and shipping. Also, the carrier pieces of this invention are more compact and manageable than the Petri dishes with the preformed gel coat. The carrier piece can be conveniently packed for storage in small sealed tubes or containers. Likewise, due to the compactness of the carrier pieces, they may be easily and inexpensively sterilized by one of several known means, avoiding the contamination hazards described with respect to prior methods. Since there is no pre-formulated gel component, the deleterious effects the change in temperature has on the water contained in the Petri dish-and-gel package are also, therefore, avoided.

The preferred gelling agent for the growth medium is a low methoxyl pectin, and compounds producing divalent metallic ions are specific in causing gelling of low methoxyl pectin. The main divalent chemical elements causing solidification are Calcium and Iron. Compounds of Calcium or Iron, therefore, which are at least somewhat soluble in aqueous solutions, such as the growth medium, may serve as hardeners for this particular gelling agent. Hence, Calcium or iron chloride, Calcium or iron phosphates, Calcium or iron sulfates, et cetera may be potential hardeners for gelling agents that are solidified by divalent metallic ions. Skilled technicians are aware that there are other gelling agents that may be effective with these, most notably various alginate gums. Likewise, there are other gelling agents that solidify when complexed with other compounds. For example, certain carrageenans solidify when mixed with Potassium chloride or with other potassium compounds.

The ratio of hardener to spreader-adhesive used for forming the reactant material solution varies depending upon the size of the finished carrier piece(s) and the particular reactant material(s) used. Generally, it is calculated that for the gelling of every 1 mL of growth medium/test sample mixture to be produced, for example, there should be available about 4-8 mg of Calcium Chloride dihydrate. In one specific example, if the growth medium/test sample mixture has a volume of 10 mL, and the growth medium/test sample mixture is dispensed in a cylindrical container having an inside diameter of 15 mm, the size and shape of the carrier piece should measure approximately 14 mm×70 mm and contain at least 40 mg of Calcium Chloride dispersed evenly over its two surfaces in order for complete solidification of the growth medium/test sample to occur when the carrier piece is inserted into the tube containing the liquid growth medium/test sample mixture.

Clearly, there is no single formula that must be used as the reactant material base solution for applying or coating the reactant material on/into the carrier piece(s). In the context of the above example, but not to be limiting with respect to the particular formulation of reactant material solution and application of the same to the carrier piece—in the above example, the reactant material is the hardener—the amount of active reactant material in the formula may vary according to the volume and thickness of the coating layers that are applied. The amount of reactant material in this example must, therefore, be determined in conjunction with the size of the carrier piece and the volume of the liquid growth medium/test sample mixture to be used in the particular test method.

Intuitively, the example discussed above represents the most basic carrier piece and reactant material formulation. There are numerous other materials from which to make the carrier piece provided, however, the particular material provides the chemical and physical properties desirable with respect to, for example, the rigidity, solubility, opaqueness, color, refractive index, absorption, and so on of the carrier piece. While the preferred materials from which to form the carrier piece are those which are clear and approach the same or similar refractive index value as the liquid growth medium or growth medium/test sample mixture, there may be test applications where it's advantageous to use an opaque or translucent material, or a colored material. Thus, the invention should not be limited in this manner.

Materials from which the carrier piece may be formed include, but are not limited to the following: paper and similar base products and combinations thereof, plastics, gums, fabrics, including polyester, filter or mesh materials, and acetate or cellulose ester film materials, among other things. The carrier piece of the invention is meant to include, for example, a soluble material dehydrated and treated such that it dissolves when contacted with the medium or medium/test sample mixture.

For a first example of a carrier piece and accompanying test application, if the fluorogenic enzyme substrate MU-gluc is incorporated as the reactant material carried by the carrier piece, and MU-gluc is used in a medium to test for the presence (or absence) of E. coli, the medium will remain liquid upon adding the carrier piece, and E. coli will grow throughout the liquid medium if present in the test sample, so that the entire medium will fluoresce under a long wave UV light. In this example, a transparent carrier piece of the invention provides the benefit of delivering a test-specific reactant material(s) into the medium in a deliberate and controlled manner without visually obscuring the sample.

In a second example, if the carrier piece carries a catalyst for the gelling agent in the liquid growth medium as well as the MU-gluc, upon adding the carrier piece to the liquid, accretion will occur to form a gel within which colony-forming units of E. coli will grow as dots that will each fluoresce when exposed to a long wave UV light. The E. coli bodies may thus be counted which was not an objective in the first example.

Many other beneficial culture media and test method combinations are possible for application with this novel carrier piece and method invention. Whenever the carrier piece carries a reactant material that is a catalyst for the gelling agent, for example, a solid, smooth accretion layer surrounding and attached to the carrier piece will result. This carrier piece-accrete complex can be removed from the growth medium or growth medium/test sample mixture and incubated to effect the growth of colony forming units, which may then be identified and counted as desired.

The following six steps are an example of a general procedure of using the method of the invention wherein the liquid growth medium includes a gelling agent, and the reactant material is a phase change material or catalyst in or on the carrier piece capable of changing the liquid growth medium from a liquid phase to a substantially solid or gel phase when the carrier piece is added to the liquid growth medium.

Step 1: The gelling agent/liquid growth medium complex is made in a container large enough for the easy introduction of one or more carrier pieces.

The gelling agents in the gelling agent/liquid growth medium complex are types that will remain in solution until reacting with the catalyst migrating from the carrier piece. Examples of such groups of gelling agents that may be suitable are Alginates, Carrageenans and Pectins. Certain types of each of these groups will solidify in the presence of certain metallic ions (such as iron or calcium), which may serve as the catalyst carried by the carrier piece.

The growth medium generally is a mix of nutrients such as peptones, sugars, and various salts, or may be more refined and contain pure chemical compounds as ingredients. In any case, the growth medium is designed to serve as a nutrient complex favoring the target organisms or cells that may be found in the test sample. The growth medium may also contain specific inhibitors such as antibiotics, bile salts and related compounds, and surfactants to prevent the growth of certain organisms. The liquid growth medium may also contain buffers or compounds to control the pH.

The carrier piece may consist of virtually any inert (non-)absorbent or (non-)porous material. There are many paper products which may suffice, as well as fiber glass or plastic materials, as above-mentioned. The size and shape of the carrier piece may vary, dependent upon the desired volume and configuration of the accretion layer(s).

The carrier piece may be of different colors to enhance the visibility of colony-forming units that appear in the accretion layers. For example, if the target organisms will grow as dark or colored colonies, the carrier piece may best be white, while if the colony units grow as colorless or light colored, a black piece may be preferable. Additionally, as above-referenced, the carrier piece may be transparent so that it is possible to see through both the carrier piece and any accumulated accretion layer(s) during subsequent analysis.

While in some applications the chemical or physical properties of the carrier piece may dictate that it not provide support to the growth medium and may even dissolve, the carrier piece should be sufficiently rigid to retain its integrity without excessive distortion when handled. Preferably, in certain applications, the carrier piece should also be able to support the weight of the accreted medium with easy handling. In this example, the carrier piece must also be able to absorb a sufficient amount of catalyst and other materials to assure sufficient supply of these to both cause the proper accretion and ingredients in the accreted material. Reactant materials that may be incorporated into or onto the carrier piece (besides the catalyst) may include nutrients (vitamins, trace metals), inhibitors (antibiotics, toxins, bile salts) and enzyme substrates, among other things.

Step 2: The test sample is added to the liquid nutrient growth medium and swirled to mix well. The test sample generally consists of an aqueous blend of a specific product, such as food, tissue, a liquid product, or a water sample. The test sample may or may not contain target organisms or other viable organisms or cells. The make-up of the test sample is generally not important to the principles of this method as long as the sample does not contain detrimental materials that will damage the integrity or concentration of the medium.

Step 3: The carrier piece is then introduced into the container holding the liquid nutrient growth medium/test sample mixture and allowed to remain, during which time the liquid growth medium (with gelling agent)/test sample mixture accumulates, or accretes, on both surfaces of the carrier piece as a result of the reaction of the catalyst with the gelling agent complex. A solid, smooth gel layer containing both the gelling agent growth medium complex and microbes from the test sample is formed.

Under some circumstances, it may be desirable to allow the accretion to continue to the inside surface(s) of the container, so that the entire volume in the container becomes gelled. In this case, this complex is placed in an incubator, and step 4 is eliminated.

Step 4: At the end of the accretion time, the carrier piece with its double layer of gelling agent growth medium/test sample complex is removed with sterile forceps and transferred into an incubation container.

Step 5: The incubation container is then placed in an incubator for a predetermined length of time.

Step 6: Following incubation, the gel accretion is examined and the colony forming units are counted and recorded from both sides of the carrier piece.

Depending upon the volume of the gelling agent growth medium/test sample complex, the number of colony forming units can be determined per ml or gm of original test sample. The carrier piece and media culture preparation method provides a simple, accurate and inexpensive way to prepare culture media for conducting microbiological analysis with an absolute minimum of facilities, extraneous equipment and time consuming procedures. The growth medium, the carrier piece with the appropriate reactant material, and the test sample can be easily transported as a kit and used most anywhere. Material costs are low, and shipping and handling costs are minimal.

While the invention has been described in detail in the foregoing description, the same is to be considered illustrative and not restrictive in character. It is understood that the examples have been described in the foregoing specification in satisfaction of the best mode and enablement requirements. It is understood that one of ordinary skill in the art could readily make a nearly infinite number of insubstantial changes and modifications to the above-described examples, and that it would be impractical to attempt to describe all such variations in the present specification. Accordingly, it is understood that all changes and modifications that come within the spirit of the invention are desired to be protected.

What is claimed is:

1. A method for preparing culture media for cultivating microorganisms comprising:
providing an aqueous growth medium including at least nutrients and a gelling agent;
providing a carrier piece separate from the medium and including a reactant material; and
contacting the medium with the carrier piece, the reactant material chemically reacting
with at least one component of the medium when the carrier piece contacts the
medium to form at least a solid growth medium.

2. The method of claim 1, wherein the reactant material includes a hardener.

3. The method of claim 1, wherein the solid growth medium contains no reflective particles, substrates, air pockets or other visually obfuscating matter so that the visual acuity of the solid growth medium is optimal.

4. A method according to claim 1, including the step of mixing a test sample with the medium to produce a mixture and then contacting the mixture with the carrier piece.

5. A method according to claim 1, including the step of allowing the carrier piece to remain in said medium so that the medium accretes on a surface of the carrier piece.

6. A method according to claim 4, including the step of allowing the carrier piece to remain in said mixture so that the medium and test sample mixture accretes on a surface of the carrier piece.

7. A method according to claim 4, wherein the method is performed at room temperature.

8. A method according to claim 1 wherein the method is performed at room temperature.

9. The method of claim 1, wherein the carrier piece is formed from a transparent material so that the carrier piece does not interfere with the visual acuity of the solid growth medium.

10. The method of claim 1, wherein the carrier piece is formed from a piece of soluble gum or resin so that the carrier piece dissolves and does not interfere with the visual acuity of the solid growth medium.

11. The method of claim 1, wherein said carrier piece dissolves when added to the growth medium.

12. A carrier piece according to claim 1.

13. A carrier piece according to claim 2.

14. A carrier piece according to claim 7.

15. A carrier piece according to claim 8.

16. A carrier piece according to claim 9.

17. A carrier piece according to claim 10.

18. A carrier piece according to claim 11.

19. A carrier piece including a reactant material for contacting with an aqueous growth medium, the reactant material chemically reacting with at least a component of the aqueous growth medium to form at least a solid growth medium, the carrier piece is formed from a material selected from the group consisting of paper, plastic, gum, fabric, glass, fiberglass, or acetate.

20. A kit for preparing a test culture medium for cultivating microorganisms, the kit comprising:
a growth medium including at least nutrients and a gelling agent; and
at least one carrier piece including a reactant material, said reactant material is capable of chemically reacting with at least one component of the growth medium when the growth medium is in solution and when the carrier piece contacts the medium to form at least a solid growth medium throughout which microorganisms may grow.

21. A kit according to claim 20, wherein the growth medium is an aqueous solution.

22. A kit according to claim 21, including a container for containing the medium.
